(19) United States
(12) Patent Application Publication
(10) Pub. No.: US 2017/0095209 A1
(43) Pub. Date: Apr. 6, 2017

(54) SENSOR MOUNT FOR A REFLECTIVE PHOTO-OPTIC SENSOR

(71) Applicant: Intelomed, Inc., Wexford, PA (US)

(72) Inventors: Helene Colaizzi, Wexford, PA (US); Thomas P. Joyce, Wexford, PA (US); Anne Brumfield, Cranberry Township, PA (US)

(21) Appl. No.: 15/284,093

(22) Filed: Oct. 3, 2016

Related U.S. Application Data
(60) Provisional application No. 62/236,628, filed on Oct. 2, 2015.

(51) Int. Cl. A61B 5/00; A61B 5/024

(52) U.S. Cl. CPC A61B 5/6833; A61B 5/02416; A61B 5/02438

(57) ABSTRACT

A holder for securing a sensor against a skin surface includes a body element made of a foam material. The body element has a recess formed therein configured for receiving and containing a sensor. A backing element is positioned proximate a face surface of the body element for closing the recess. An adhesive element is coupled with a face surface of the body element opposite the backing element for adhering the body element to a skin surface so a sensor contained therein presses against the skin surface. A back pad made of a foam material is configured for being positioned in the recess to underlie a sensor received in the recess. The back pad is configured to be compressed within the recess when the body element is adhered to a skin surface to provide a contact force for directing the sensor against the skin surface.

### CROSS-REFERENCE TO RELATED APPLICATION

This application claims the benefit of U.S. Provisional Patent Application Ser. No. 62/236,628 filed on Oct. 2, 2015, the disclosure of which is expressly incorporated by reference herein in its entirety.

### FIELD OF THE INVENTION

The present invention relates generally to sensor devices for measuring biological signals and more specifically to a holder device for securing a sensor to a skin surface of a person for signal acquisition.

### BACKGROUND OF THE INVENTION

Sensors are used in a variety of different locations and manners with and on the human body for testing and diagnosing one or more of the systems related to the current state of a living body. Such sensors are generally used for obtaining a biological signal, of some type, from the human body. The signals are then used or otherwise processed to provide an indication of the operation of such biological systems. Oftentimes, such sensors are external, non-invasive sensors for obtaining biological signals from outside the body and the signals are then directed to a processing device, such as a computer. For example, when testing the cardiovascular system, one or more sensors may be placed externally on various parts of the body to obtain a biological signal that is indicative of the current state of that body's cardiovascular system.

One type of external and non-invasive sensor is a photo-optic sensor that uses the emission and collection of light signals to measure certain systems. Specifically, a light signal of a certain wavelength is emitted from a light source, such as a light emitting diode (LED) onto the skin surface of a person. The light is both absorbed and reflected by the body, including by the tissue of the body as well as the blood flowing in the arteries. A detector then detects the transmitted and/or reflected light. The amount of light passing through a portion of the body or the amount of light that is reflected and the various frequencies of such light signals may be evaluated to generate a biological electrical signal that is indicative of certain conditions of the measured system in the body. In reflective photo-optic sensors, the emission of light signals and the collection of reflected signals are used to evaluate the measured system. During testing, a reflective photo-optic sensor may be used for photo-plethysmography measurements, oximetry measurements, or both clinical measurements, and may be performed by the same sensor or multiple sensors.

For such sensor applications, the sensor is generally held in place on the body, such as against the skin, in some fashion, often depending upon the kind of signal that is to be detected. It is important that the sensor is held in place in a proper and stable manner at the selected location, in order to ensure that an optimal signal is obtained. As may be appreciated, a photo-optic sensor that is detecting the flow of blood within a vessel is affected by the engagement of the sensor with the skin proximate to the underlying vasculature that is providing the signal. If the sensor is held in contact improperly or unevenly, the quality of the signal that is obtained may be compromised.

Furthermore, since a person may be wearing the sensor and sensor holder for an extended period of time, the comfort of the wearer is also important. Discomfort may lead the person wearing the sensor and sensor holder to try and move the sensor or readjust the sensor holder. Such movement can lead to a degradation in the signal acquisition process and the quality of a sensed signal, as well as an overall degradation of the evaluation of the body system being monitored.

As noted, a sensor should be secured to provide proper contact of the sensor to the skin in order to capture the desired biological signal with good signal integrity and consistency. As such, movement of the sensor on the body should be minimized. A sensor should be secured in position to reduce any detrimental effects attributable to the motion of the wearer during clinical use. If the sensor is held to loosely or too tightly, the quality of the signal that is obtained may be compromised. For example, a sensor placement that is too loose may result in an intermittent signal or a degraded signal quality due to inconsistent contact of the sensor with the skin. Conversely, a sensor held too tightly to the skin may detrimentally affect the underlying vasculature, as well as degrade the quality or consistency of the resultant signal that is captured. For some biological signals, the signal integrity is not as critical. However, for more sophisticated diagnostics wherein the biological signal will be further processed and analyzed, including for example frequency processing and time dependent comparisons, the sensor must be maintained properly in both position and contact force for the duration of the diagnostic to be useful.

Some currently available means of securing a sensor involve simply taping the sensor to the area with medical grade adhesive tape. Other techniques involve sensor holders that are simply flexible adhesive strip structures that are stuck against the skin or wrapped around an appendage, such as a finger, to contain the sensor. Other sensor holders are typically constructed of rigid plastics that require expensive pre-production form molding or machining to produce. For example, finger clip sensors/sensor holders are used for gripping a finger to obtain a signal, but are not suitable for other areas of the body. Additionally, some sensor holder designs also use elastic bands to secure the sensor to the application site, such as the head, or an appendage. However, such a means for affixing the sensor is often not optimal from the standpoint of removal of undesired motion artifacts. For example, the impression on the skin resulting from the pressure of the plastic or size of the elastic band may not be acceptable or comfortable to the wearer of the sensor. Still further, such elaborate sensor holder systems are expensive to produce.

At the same time, a suitable sensor holder must achieve all of these requirements while addressing needed economies to be acceptable to the marketplace. This is particularly so in the current medical industry economy wherein cost is a significant factor. In many scenarios, the sensor holder will be disposable and so a complicated and expensive apparatus is not desirable. At the same time, cheap sensor holders may not be suitable for the more sophisticated measurements and processing.

Accordingly, a need still exists for a disposable and cost effective sensor holder, constructed of materials that are biocompatible with human skin, and that is capable of maintaining a sensor in a proper location for the acquisition of a biological signal. There is a further need for a sensor holder that may be comfortably worn for a long period of time while still maintaining sufficient and uniform contact of the sensor with a body surface to ensure robust and consistent biological signals. There is a further need of such a sensor holder for use with a photo-optic sensor to ensure quality biological signals for further processing. These needs and other needs in the art are met with a sensor holder as described herein.

### SUMMARY OF THE INVENTION

A holder for securing a sensor against a skin surface includes a body element made of a foam material. The body element has a recess formed therein that is configured for receiving and containing a sensor. A backing element is positioned proximate to a face surface of the body element and closes the recess at one end thereof. An adhesive element is coupled with a face surface of the body element opposite the backing element. The adhesive element is configured for adhering the body element to a skin surface so that a sensor that is contained therein presses against the skin surface. A back pad made of a foam material is configured for being positioned in the recess in the body element. The back pad underlies a sensor received in the recess. The back pad is configured to be compressed within the recess when the body element is adhered to a skin surface. The back pad and sensor holder provide a contact force for directing the sensor against the skin surface.

### BRIEF DESCRIPTION OF THE DRAWINGS

The accompanying drawings, which are incorporated in and constitute a part of this specification, illustrate embodiments of the invention and, together with a general description of the invention given below, serve to explain the principles of the invention. Aspects, features, benefits and advantages of the embodiments described herein will be apparent with regard to the following description, appended claims and accompanying drawings where:

FIG. 1 illustrates a perspective view of a sensor holder including a sensor according to an embodiment of the invention.

FIG. 2 illustrates an exploded view of the sensor holder of FIG. 1.

FIGS. 3-3A are cross-sectional profile views of a sensor holder according to an embodiment of the invention showing the sensor holder with the sensor and installed on a skin surface.

FIG. 4 illustrates an exploded view of an alternative embodiment of a sensor holder of the invention.

FIGS. 4A is cross-sectional profile view of the sensor holder of FIG. 4 showing the sensor holder with the sensor.

FIG. 5 illustrates an exploded view of an alternative embodiment of a sensor holder of the invention.

FIGS. 5A is cross-sectional profile view of the sensor holder of FIG. 4 showing the sensor holder with the sensor.

FIGS. 6A-6D illustrate individual components of the sensor holder of FIG. 1 according to an embodiment of the invention.

FIG. 7 illustrates an exploded view of an alternative embodiment of a sensor holder of the invention.

### DETAILED DESCRIPTION OF EMBODIMENTS OF THE INVENTION

The present application addresses the drawbacks and inherent shortcomings of existing sensor holder designs from the standpoint of the specific mechanical design and the materials employed to address signal quality, comfort, biocompatibility and the economies desired. The embodiments of the invention are particularly useful for

A novel sensor holder device is described herein that is constructed from a composite of closed-cell foam elements that are light weight and that cooperate to provide a suitable containment and application of a sensor. The foam elements are configured to both contain the sensor and apply an appropriate engagement force on the sensor to the external cutaneous site or skin surface on which the sensor is used. In one aspect of the invention, the sensor holder device is suitable for use with a photo-optic sensor, such as one that is used to obtain a photoplethysmogram signal, “pleth” signal or PPG signal. A PPG signal is a biological signal optically obtained using a sensor similar to that used for obtaining a pulse oximetry reading. A photo-optic sensor such as a reflective sensor may be incorporated into the sensor holder device, and the sensor holder may be sized and shaped accordingly.

However, it should be noted that the dimensions of the sensor holder may be altered to accommodate a sensor substrate of a different size. Also, the holder device of the invention may be used to secure other types of sensors, such as pressure sensors, strain gauges, ultrasound devices, electrical impedance measurement devices, electro-cardiogram devices, etc. Therefore, the invention is not limited to the type of sensor that is secured. A biocompatible surface adhesive is used with an outer foam element or component and provides suitable securing of the holder device to a cutaneous or skin surface. The overall construction and arrangement of the components provides a suitable pressure exerted on the sensor and skin site over a desired duration, while minimizing signal artifacts from lateral movement. The properties of the sensor holder device combined to distribute compressive forces over an area to provide optimal wearer comfort while minimizing the anatomical site required. In one embodiment of the invention, an outer, hard plastic layer is utilized with the composite foam elements, opposite the adhesive layer, for applying a pressure on the adhesive layer when the sensor holder device is placed to secure the holder device, while protecting the foam elements.

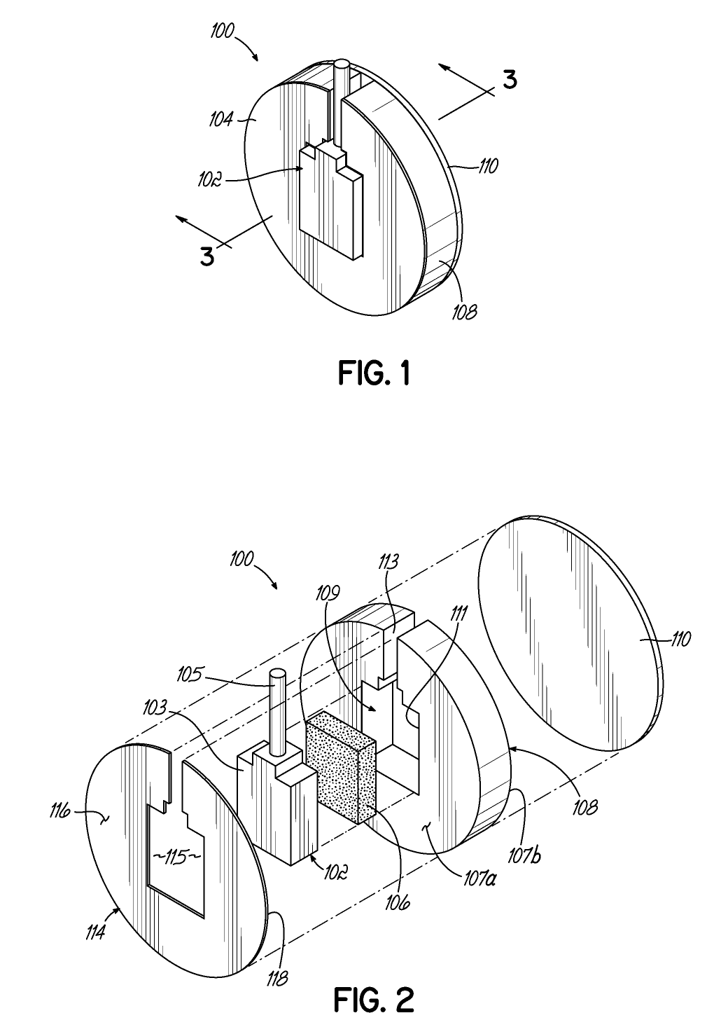

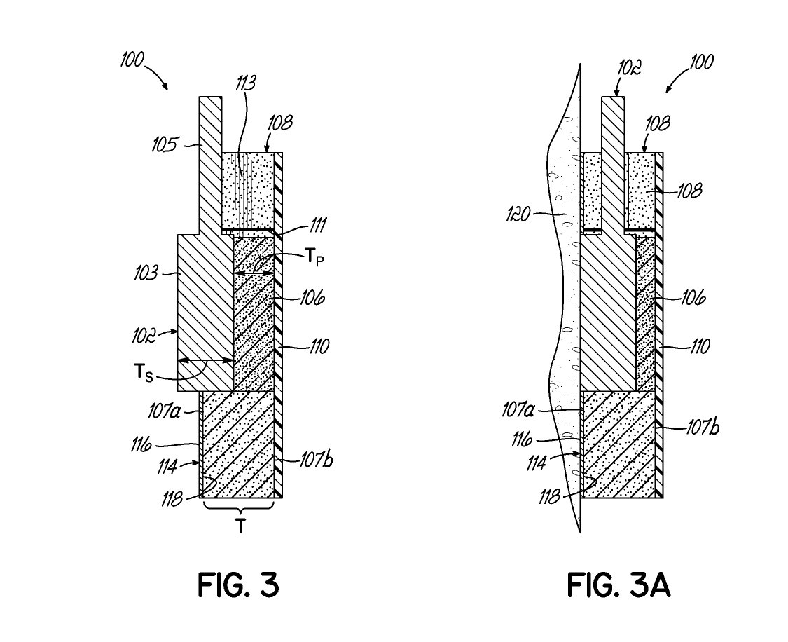

FIG. 1 illustrates a perspective profile view of a sensor holder device or sensor holder 100 according to one embodiment of the invention. The sensor holder 100, as illustrated in the Figures, is configured to operate in conjunction with a generally flat and rectangular sensor 102. One possible sensor for use with the invention is the Nonin 8000R Reflective Pulse Oximeter Sensor from Nonin Medical, Inc. of Plymouth Minn. The sensor holder 100 is configured such that, when the sensor holder is placed on a skin surface of a patient, the sensor directly contacts the skin and is held in place with a desirable force. (See FIG. 3A). While the sensor 102 is illustrated having a rectangular cross-section, sensors of other shapes may be used with the invention and the recess 109 and back pad 106 of the sensor holder, as discussed herein, can be shaped accordingly to accept other sensors and shapes.

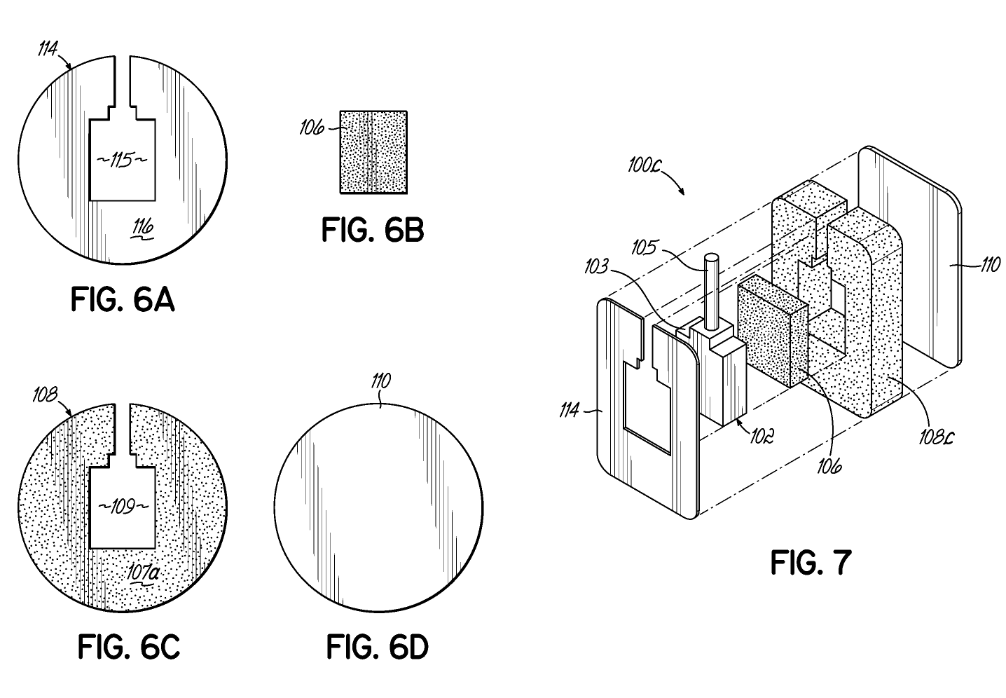

The sensor holder device 100 of FIG. 2 includes foam elements 106, 108 that cooperate together to both contain the sensor and position the sensor with respect to the adhesive layer in order to have a uniform application of force around the sensor against the skin to direct the sensor against the skin surface for obtaining a biological signal with good signal quality and integrity. More specifically, the sensor holder 100 includes a body element 108 that is made of a suitable foam to provide structure to the body element. The foam of the body element is formed to create and define the outer shape of the sensor holder 100 as seen in FIG. 1. The body element 108 may have a round cross-section configuration as illustrated in FIGS. 1-2 or a rectangular cross-section configuration as shown in FIG. 6. In one preferred embodiment, the sensor holder has a contact area in the range of 1-2 in². For example, a circular body element as disclosed may have a diameter of 1-2 inches. A rectangular body element may have dimensions of around 1.00×1.5 inches. The body element 108 is formed or machined from a sheet of foam material and is configured to define a cavity or recess 109 therein. The recess 109 is sized and configured for containing both the sensor 102 and a foam back pad 106 that lies behind the sensor 102 in accordance with an embodiment of the invention. The body element may have a thickness T in the range of 0.125-0.75 inches, although such a dimension is not limiting to the invention. In one embodiment, the thickness of the foam of the body element 108 is around 0.313 inches or 5/16 inches.

A recess 109 may be formed in the body element and configured to contain a selected sensor 102. The recess might be shaped appropriately depending on the outer shape and dimensions of the sensor 102, such as the Nonin 8000R Sensor. In one embodiment, the recess 109 may have dimensions of generally 0.6000×0.440 inches. Also, the recess 109 will generally be somewhat centered in the body element 108 to ensure that a sensor will be equally held and secured around its entire periphery for proper signal acquisition. The recess is constructed and dimensioned to allow minimal play for the sensor employed. The sensor 102, as shown in FIG. 2 includes a sensor element 103 for sensing a biological signal and a cable or cable assembly for transmitting the detected signal to a processing device, such as a computer device, remote to the sensor 102 and body element 108. To that end, the recess 109 is formed to have a sensor portion 111 and a cable portion 113 so that the sensor 102 may fit generally flush into the recess 109 as shown in FIG. 3. The cable portion 113 is configured to capture the cable and direct it to extend from the side of the sensor holder in a controlled manner. In that way, the sensor holder minimizes movement and motion artifacts of the sensor that may be induced by downstream cable motion. In that way, the foam body element surrounds and contains the sensor and elements thereof around the entire sensor. In the embodiment of the sensor holder device in FIGS. 2-3, the recess is formed entirely through the body element between face surfaces or faces 107a, 107b of the body element 108.

In some embodiments of the invention as disclosed herein, the recess extends all the way through the body element 108, between face surfaces, and must be closed on one side by a backing element or backing material of some kind. In some embodiments, the backing element is a separate element that is positioned proximate to face surface 107b of the body element and adhered to the body element. In other embodiments, the backing element might be integral with the body element. For example, the recess may not be formed all the way through the body element as described herein, and so the backing material of backing element may be the additional foam thickness of the body element.

The sensor holder device 100 of FIGS. 2-3 includes a separate backing plate or capstock 110 that is positioned on one face 107b of the body element 108. The backing plate 110 preferably has the same outer shape of the body element 108 and may be adhered to the adjacent body element with a suitable adhesive material. The backing plate closes one end of the recess 109 for containing the other elements of the sensor holder, including the sensor.

The sensor holder device 100 has an adhesive element positioned on a face surface thereof for engaging the a skin surface of a user for securing the sensor holder 100. In the illustrated embodiments, the sensor holder further includes a skin-contacting adhesive material layer or adhesive sheet appropriately shaped to match the shape of the body element and surround the sensor 102. For example, a layer 114 of a suitable biocompatible adhesive may be applied to face surface 107a of the body element. Alternatively a separate sheet of adhesive, such as a double sided and adhesive-coated sheet or medical tape might be used, having one side secured to surface 107a of the body element. One suitable adhesive sheet is a 1597 double sided medical tape from 3M of St. Paul Minn. If a sheet is used, the sheet has an appropriately formed opening 115 formed therein to accommodate the sensor 102 including sensor element 103 and cable 105. In that way the sensor element and cable may be contained within the body element 108 and the sensor element 103 is exposed to the skin when the sensor holder is affixed to a skin surface. One side of the skin-contacting adhesive sheet 114 may include a first adhesive material 116 configured to provide an adhesive surface for holding the sensor holder device 100 against the skin surface, thereby maintaining physical contact between the skin and the sensor. The adhesive material 116 should be a type not irritating to the skin. The skin-contacting first adhesive on the adhesive sheet 114 may be selected based upon a desired hold time. In accordance with one embodiment, a hold time of from 30 minutes up to six hours may be desirable. Additionally, the skin-contacting adhesive sheet 104 includes an additional second adhesive 118 on an opposite side from the skin-contacting adhesive for holding the adhesive sheet 114 securely to the rest of the sensor holder device 100. As may be appreciated the adhesive material layer or sheet 114 may have a separate peel away layer (not shown) attached thereto to cover and protect the adhesive until it is ready for use to apply the sensor holder to a skin surface.

In one aspect of the invention, it is desirable to have an area of face surface 107a that is adhered to a skin surface that is a particular ratio with respect to the size of the sensor 102, in order to have good adhesion and a suitable force against the sensor for signal acquisition. In one embodiment, the adhesive contact area ratio with respect to a sensor for the body element 108 is in the range of 4:1 to 15:1. This will vary, as will be understood with respect to the size of the sensor used as well as the size of the sensor holder. Maintaining such a ratio in the range as noted ensures proper signal acquisition and good adherence of the sensor.

In accordance with another aspect of the invention, the sensor holder 100 includes a foam sensor back pad 106 that seats within the body element 108. Referring to FIG. 3, the sensor back pad sits behind the sensor 102 within the recess 109 and is configured with a specific foam density and compression stiffness, and is dimensioned to deliver an amount of contact force to the sensor when it is compressed in use to force it against a skin surface. The back pad 106 is compressed when the sensor holder 100 is adhered to the skin surface. Specifically, the recess 109 is formed to have a depth dimension D that cooperates with the sensor element 103 and the back pad 106 to provide a force in a desired range. The depth dimension of the recess, which may be dictated by the thickness T of the body element, is less than the combined thickness dimension T_{S} of the sensor element 103 and the thickness dimension T_{P} of the uncompressed back pad 106. Referring to FIGS. 3 and 3A, when the sensor holder 100 and sensor 102 are placed on a skin surface 120, the back pad 106 is compressed by the sensor against the backing plate 110. In that way, the contact surface of the sensor on the skin surface 120 is generally flush with the adhesive sheet 114.

The compression of the back pad allows the sensor element 103 to seat within the recess 109 and the surface of adhesive sheet 114 can contact the skin surface completely around the sensor body element 108 for proper signal acquisition. The sensor element 103 is then forced against the skin by the compressed back pad 106 to obtain a biological signal. That is, the compressed back pad 106 translates an amount of force that is applied to the back of the sensor element to drive it against the skin surface 120, maintaining the tension between the sensor and the skin. In an embodiment of the invention, the foam back pad provides a contact force on the sensor to provide a contact force or contact pressure in the range of 60-180 mm Hg against a skin surface of a wearer. This minimizes the venous contribution to the acquired signal from the application without constricting the arterial contribution. In the embodiment of FIGS. 2-3, the backing plate 110 compresses the back pad 106. To that end, the backing plate 110 may include, for example, an amount of pre-applied adhesive for adhering the backing plate to both the sensor back pad 106 and the body element 108, thereby providing, in concert with the skin-contacting adhesive sheet 104, a means for holding the sensor holder device 100 together.

In one embodiment of the invention, a closed cell foam material may be used to provide the primary structure of the body element 108. For example, closed cell foams of ethylene vinyl acetate, polyethylene, polyurethane, neoprene, polystyrene and polypropylene might be used. In one embodiment, an Ethylene Vinyl Acetate (EVA) foam material, for example, may be used due to its semi-rigidity while providing a soft feel to the skin. Other blended copolymers may also be used. One suitable foam material for the sensor holder device 100 is Volara Type EO foam that is available from Sekisui Voltek of Lawrence MA. Such a foam type might also be used for both the body element 108 and the back pad 106. In one embodiment, a combination of foam having a more rigid foam density may be used for the body element 108 (thus having less stretch) and foam having a more flexible foam density may used in for the back pad 106 (thus having more compression) in the center of the device between the sensor element 103 and the stiffer plastic of the backing plate 110 to create a spring force for providing constant tension of the sensor against the skin surface. The foam as used in the invention may have varying density as measured in pounds per cubic foot or lb/ft³.

The back pad 106 may have a thickness dimension Tp in the range of ⅛ to 3/16 inches. In one embodiment, the recess 109 might have a depth dimension that matches the thickness of the foam body element 108. Accordingly, depending on the sensor thickness, once the sensor holder device is installed on a skin surface, the back pad 106 might be compressed various different depths or percentages. Various combinations of foam density and foam dimensions may be used to achieve an optimal compression force on a sensor against a skin surface.

In one exemplary embodiment, a 6 lb/ft³ density foam might be used for forming the body element 108 with a similar 6 lb/ft³ foam used for forming the back pad 106. In such a scenario, the foam back pad 106 might utilize a thickness of ⅛ inches in some embodiments, and a thickness of 3/16 inches in other embodiments.

In another exemplary embodiment, a 6 lb/ft³ foam might be used for forming the body element 108 while a 4 lb/ft³ density foam might be used for forming the back pad 106. In such a scenario, the foam back pad 106 might utilize a thickness of ⅛ inches in some embodiments, and a thickness of 3/16 inches in other embodiments.

In still another exemplary embodiment, a 4 lb/ft³ foam might be used for forming the body element 108 while a 2 lb/ft³ density foam might be used for forming the back pad 106. In such a scenario, the foam back pad 106 might utilize a thickness of ⅛ inches in some embodiments.

For providing a desirable contact pressure range against a skin surface of a wearer, the dimensions of the foam back pad 106 and the depth of the recess 109 might be configured so that the sensor would compress the foam back pad in the range of 40-80% of the thickness of the back pad.

The backing plate 110 may be made of a suitable thermoplastic material. In one embodiment, a polyethylene terephthalate thermoplastic (e.g., PET, PETE, PETG) may function as a rigid plastic backing plate 110 for providing rigidity against the lower density foam of the back pad 106. In one particular embodiment, PETG is used. The backing plate 110 would act against the compressed back pad to create the sensor tension against the skin surface. The backing plate 110 may have a thickness in the range of 0.010-0.030 inches. In one exemplary embodiment, a backing plate having a thickness of around 0.020 inches may be used to create the rigidity desired while still enabling a degree of flexibility of the entire assembly to contour to the skin where the sensor will be applied.

FIGS. 3 and 3A illustrate the sensor holder device in use against a skin surface or other surface. The detecting surface of a sensor 102 that lies against a cutaneous or skin surface is generally flush with the adhesive element 114 and the face surface 107a of the sensor holder when the sensor holder is adhered to the skin surface. Specifically, the depth dimension of the recess and the thickness dimension of the back pad in one embodiment will project the sensor element 103 forwardly as shown in FIG. 3. Then when the adhesive layer 104 is pressed against the skin surface 120 to secure the holder device 100 and sensor 102 in position, the sensor 102 is generally flush with the face surface 107a and the back pad 106 is compressed a certain percentage, as noted herein, in order to provide the desired force on the sensor element 103 against the surface 120. As the adhesive layer 104 adheres to the surface 120, the compressed back pad 106 provides a continuous force on the sensor element against surface 120. This provides for the capture of a biological signal that has good signal integrity and consistency.

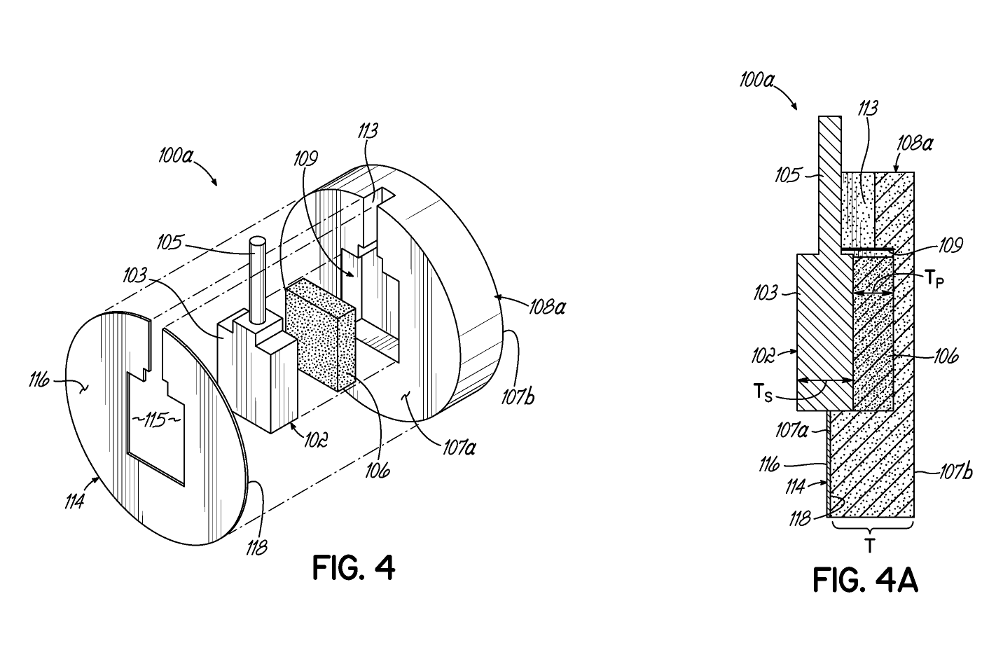

FIGS. 4 and 4A illustrate an alternative embodiment 100a of the invention wherein the backing plate 110 is eliminated and foam backing material closes the recess at one end. In the FIG. 4 embodiment, some of the foam of the body element acts as the backing material on one end of the recess 109. More specifically, the recess 109 is formed in the body element 108a but does not extend all the way through the body element. Many of the elements of the embodiment 100a are similar to the embodiment 100 as illustrated in FIGS. 1-3 and so like reference numerals are used. Specifically, the recess 109 formed in body element 108a extends into the body element but does not extend completely through between face surfaces 107a, 107b. Rather, the recess extends to a suitable depth to capture the back pad 106 and sensor 102 therein. One end or side of the recess is closed by a foam portion of the body element 108a rather than a separate backing plate 110. That is, there is no need for a separate backing plate 110 to contain the sensor and back pad 106 or to act thereon. The foam material of the body element 108a acts as backing material and acts on the compressed back pad 106 similar to the embodiment as shown in FIG. 3A. Alternatively, if a harder outer surface on the sensor holder 100a is desired, a backing plate 110 (not shown in FIGS. 4-4A) might be utilized on the face surface 107b similar to the embodiment shown in FIGS. 1-3.

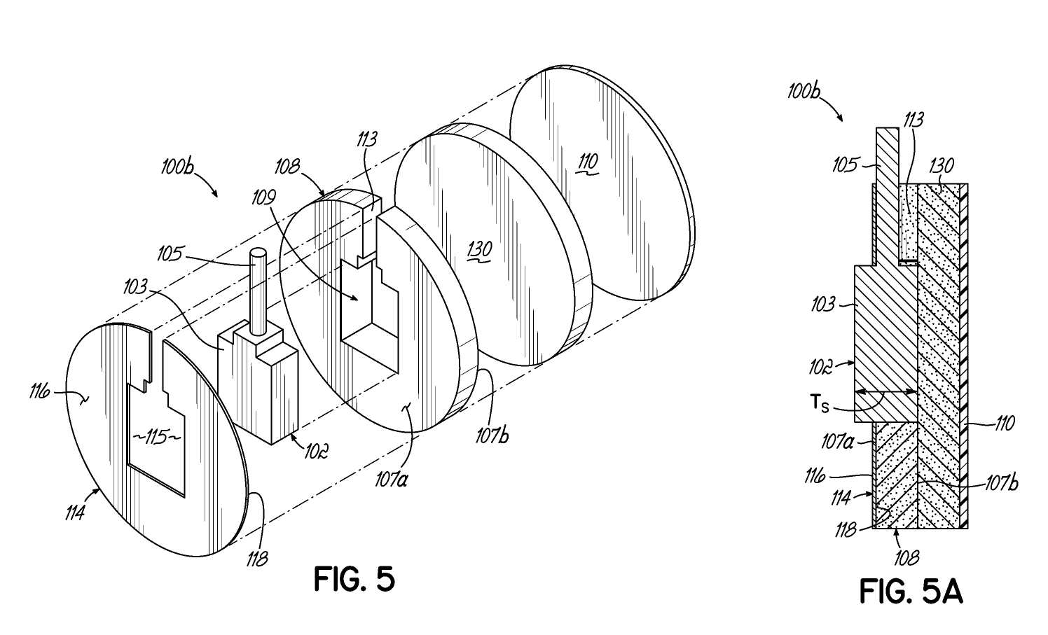

FIGS. 5-5A illustrate another embodiment of the invention. The sensor holder device 100b uses an additional and separate foam backing element in combination with a body element in order to provide a compression force on the sensor rather than a smaller inserted back pad. Specifically, as shown in FIG. 5, recess is formed through the body element 108b. Then the sensor holder device 100b has a foam backing element 130 that is adhered to the rear face surface 107b of body element 108b to close one end of recess 109 and also provide a compressive force on the sensor. The foam backing element is adhered to the body element using a suitable adhesive to keep the two elements together in a generally permanent fashion. In such an embodiment, the foam backing element 130 may be selected to provide the desired compression force, thus eliminating the need for a back pad 106. As illustrated in the Figures, in order to have a portion of the sensor element 103 extend forwardly of the face surface 107a of the sensor holder 100b before it is applied to a skin surface, and without a separate back pad 106, the body element 108b may be made thinner, so as to define a more shallow recess 109. For example, the body element might be around 3/16 inches rather than, for example 5/16 inches. The foam backing element, in one embodiment is also around 3/16 inches. When the sensor holder 100b is applied to a skin surface, the foam backing element 130 is compressed proximate the sensor 102. As such, the foam backing element 130 might be made of a suitable 4 lb/ft³ or 6 lb/ft³ foam material for providing the desired compressive force on the sensor. A separate backing plate 110 may or may not be used with such a design, although a backing plate is shown in FIGS. 5-5A.

FIGS. 6A-6D illustrate front views of several of the components of the sensor holder device 100 as described in the embodiment illustrated in various aspects of the Figures. For example, FIG. 6A illustrates the skin-contacting adhesive layer or sheet 104. As shown in FIG. 6A, the skin-contacting adhesive sheet 104 defines an opening 115 to go around a sensor 102 being inserted into the holder and in alignment with the recess 109 formed in the body element 108, thereby providing an opening through which the sensor can directly contact the patient's skin. To that end, the opening 115 may be similar in cross-sectional shape to the recess 109. FIG. 6B illustrates the sensor back pad 106. As noted above, the sensor back pad may be sized and shaped appropriately based upon the specific type of sensor that is being used and the recess 109. FIG. 6C illustrates the structural foam body element 108. The body element 108 may include a recess 109 that is shaped based upon the sensor being inserted into the holder and having a depth to accommodate both the sensor 102 and back pad 106. Generally, as shown in FIGS. 1-3, the body element 108 will have a thickness defining the recess 109. FIG. 6D illustrates the backing plate 110 for the sensor holder device 100. As noted above, the backing plate 110 may be made from a relatively rigid material such that a spring force is applied to the sensor back pad 106 by the backing plate when the back pad 106 is compressed, thereby applying the spring force to the sensor itself.

FIG. 7 illustrates another embodiment of the sensor that has a different shape or form factor. For example, the sensor holder device 100c has a body element 108c that has a rectangular cross-section. Various aspects of the other elements of the sensor holder 100c are similar to the elements as discussed herein with respect to other figures and so like reference numerals are used.

It should be noted that the relative thicknesses, sizes and positions of the components of sensor holder device 100 as are shown in the Figures are by way of example only. As noted above, depending upon the type and size of the photo-optic sensor being used, the dimensions of the individual components of the sensor holder device 100 may vary accordingly. For example, the recess as defined by the structural foam 108 may be sized differently for an alternate sensor. Similarly, the size and shape of back pad 106 may be altered if another sensor is used.

During, for example, a cardiovascular stress test, the sensor and sensor holder device may be placed near the center of the forehead to capture changes in light from a branch of the external carotid artery. The external carotid artery captures the dynamic changes in the signal that are more consistent with the lower body as opposed to the placement above the eyebrow that captures changes from a branch of the internal carotid artery that are more closely aligned with the brain.

While the present invention has been illustrated by the description of the embodiments thereof, and while the embodiments have been described in considerable detail, it is not the intention of the Applicants to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of Applicants' general inventive concept.

Various aspects of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

It is also to be understood that the terminology used in the description is for the purpose of describing the particular versions or embodiments only, and is not intended to limit the scope of the invention.

It must also be noted that as used herein and in the appended claims, the singular forms “a,” “an,” and “the” include plural references unless the context clearly dictates otherwise. Thus, for example, reference to a “sensor” is a reference to one or more sensors and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are described. All publications mentioned herein are incorporated by reference. Nothing herein is to be construed as an admission that the embodiments described herein are not entitled to antedate such disclosure by virtue of prior invention.

What is claimed is:

1. A holder for securing a sensor against a skin surface, comprising:
a body element made of a foam material, the body element having a recess formed therein configured for receiving and containing a sensor;
a backing element positioned proximate a face surface of the body element for closing the recess at one end thereof;
an adhesive element coupled with a face surface of the body element opposite the backing element, the adhesive element configured for adhering the body element to a skin surface so a sensor contained therein presses against the skin surface;
a back pad made of a foam material, the back pad configured for being positioned in the recess in the body element to underlie a sensor received in the recess;
the back pad configured to be compressed within the recess when the body element is adhered to a skin surface and to provide a contact force for directing the sensor against the skin surface.

2. The holder of claim 1 wherein the backing element includes a separate element adhered to the face surface of the body element for closing the recess.

3. The holder of claim 2 wherein the backing element is a plastic backing plate.

4. The holder of claim 2 wherein the backing element is a foam element.

5. The holder of claim 1 wherein the backing element is integral with the body element and closes the recess at one end thereof.

6. The holder of claim 1 wherein the foam material of the body element has a density that is the same as the density of the foam material of the back pad.

7. The holder of claim 1 wherein the foam material of the body element has a density that is higher than the density of the foam material of the back pad.

8. The holder of claim 1 wherein the adhesive element is a double sided and adhesive-coated sheet of material.

9. The holder of claim 1 wherein the back pad is made of a foam material configured for providing a contact force in the range of 60-180 mm Hg when compressed for directing the sensor against the skin surface.

9. The holder of claim 1 wherein the back pad is made of a foam material configured for providing a contact force in the range of 60-180 mm Hg when compressed for directing the sensor against the skin surface.

10. The holder of claim 1 wherein the back pad and the recess are configured so the back pad is compressed in the range of 40-80% of a thickness of the back pad when the body element is adhered to a skin surface.
